(19) United States
(12) Patent Application Publication
(10) Pub. No.: US 2016/0158398 A1
(43) Pub. Date: Jun. 9, 2016

(54) Fragrance Dispensing Apparatus

(71) Applicant: Michael Glenn Jones, San Diego, CA (US)

(72) Inventor: Michael Glenn Jones, San Diego, CA (US)

(21) Appl. No.: 14/949,594

(22) Filed: Nov. 23, 2015

Related U.S. Application Data
(60) Provisional application No. 62/089,529, filed on Dec. 9, 2014.

(51) Int. Cl. A61L 9/12

(52) U.S. Cl. CPC A61L 9/122; A61L 9/127

(57) ABSTRACT

An apparatus for dispersing an aromatic fragrance includes an oscillating fan, a stand, a base, a fragrance dispenser, and a fragrance container. The fragrance dispensing apparatus is used to evenly and effectively disperse a fragrance throughout a room or household.

### CROSS-REFERENCE TO RELATED APPLICATION

The present application claims the benefit of U.S. Provisional Patent Application No. 62/089,529, filed on Dec. 9, 2014, entitled “Fan Fragrance”, the entire invention of which is incorporated by reference herein.

### FIELD OF THE INVENTION

The present invention is directed to automatic air fresheners and oscillating fans. More specifically, the present invention relates to oscillating fans with attached fragrance dispenser for keeping rooms smelling fresh and clean.

### BACKGROUND

Often times, it can be difficult for homeowners to keep a house smelling fresh and clean. These troubles can be attributed to pet odors, cigarette smoke, mold buildup, or even odors that come from simply living in the house. Common methods to combating these odors include using aerosol sprays, gels and plug-ins. The downside to these methods is that they do not adequately disperse a fragrance throughout an entire room. While sprays may be applied to multiple areas of a house, their fragrance diminishes relatively quickly. As for gels and plug-ins, their fragrances are more lasting, but only affect a small area of a room. Multiple gels or plug-ins are needed to keep an entire household smelling fresh.

Accordingly, there is present need for an air freshening system that can be used to automatically disperse a fragrance throughout an entire room or household while also maintaining some level of potency.

### SUMMARY OF THE INVENTION

The present invention, in its many embodiments, provides an apparatus that is used to evenly and effectively disperse a fragrance throughout a room or household. Because this invention uses a fan, it can also be used as a cooling device such as cooling the inhabitants of a household. The present invention uses a fragrance dispenser, which is attached to a fan, to release controlled amounts of fragrance into the air. The fan is used to improve and control the rate of the dispersion of the fragrance through a house.

Additionally, the fan and the fragrance can be controlled independently to easily regulate how much air the fan can blow or how much fragrance is released by the fragrance dispenser.

In an exemplary embodiment, the present invention is an apparatus for dispersing an aromatic fragrance, comprising a fan, a stand, a base, a fragrance dispenser, and a fragrance container, wherein the oscillating fan is removably connected to the stand, and fragrance dispenser is removably connected to the oscillating fan. In another embodiment, the fragrance container is removably connected to the fragrance dispenser, wherein the fragrance container and the fragrance dispenser are in fluid communication.

In a further embodiment, the fragrance dispenser comprises a fragrance dispensing unit, wherein fragrance dispensing unit further comprises a body, a fragrance channel, a heating element, and one or more vents. In yet another embodiment, the fragrance container further comprises a body, a fragrance chamber, a container opening, and a wick.

In still another embodiment, the oscillating fan further comprises one or more rotating blades surrounded by a cage, wherein the cage prevents any object from contacting rotating blades or interfering with the motion of rotating blades, and an oscillator mechanism extending from the top of fan, wherein oscillator mechanism allows a user to control the oscillation of oscillating fan by manipulating a toggle switch.

In yet another embodiment, the stand further comprises a mobile, tubular lower frame member having two ends, wherein one of the ends is attached to the oscillating fan with a hinged connection, and wherein other end of tubular frame member is attached to a base, to vertically support the fan and maintain the fan in a balanced position. In a further embodiment, the stand further comprises a plurality of buttons for controlling the flow of electrical power to oscillating fan, the speed of oscillating fan, the function of oscillating fan, and the function of fragrance dispensing unit. In one embodiment, the body of fragrance dispenser further comprises housing for a heating element, and the housing is attached to left side of oscillating fan.

In one exemplary embodiment, the fragrance channel comprises an opening through the center of the body of fragrance dispenser, creating an opening on the overhanging portion of body to connect the fragrance container to the fragrance dispensing unit, and the fragrance channel further allows fragrance to travel to the vents of fragrance dispenser. In yet another embodiment, the vents of the fragrance dispensing unit are located on top of the body of fragrance dispensing unit, wherein the vents further comprise small holes to allow dispersal of a fragrance gas created by heating element of the fragrance dispensing unit. In still another embodiment, the body of fragrance container is removably connected to the fragrance dispenser via the fragrance channel.

In one embodiment, the fragrance chamber comprises a hollowed-out portion of the body of the fragrance container for storing a fragrance substance. The wick comprises packed fibers extending from the fragrance chamber, and wherein the wick further protrudes from an opening in the fragrance container to direct the fragrance substance out of the fragrance chamber and into the fragrance dispensing unit through capillary action.

### DESCRIPTION OF THE FIGURES

Embodiments of the invention will now be described, by way of example only, with reference to the accompanying figures.

FIG. 1 illustrates a perspective view of an exemplary embodiment of a fragrance dispensing apparatus in accordance with the principles of the present invention;

FIG. 2 illustrates a rear perspective view of an exemplary embodiment of a fragrance dispensing apparatus in accordance with the principles of the present invention;

FIG. 3 illustrates a side perspective view of an exemplary embodiment of a fragrance dispensing apparatus in accordance with the principles of the present invention;

FIG. 3 illustrates a rear perspective view of an exemplary embodiment of a fragrance dispensing apparatus in accordance with the principles of the present invention;

FIG. 4 illustrates a rear perspective view of an exemplary embodiment of a fragrance dispensing apparatus, with the fragrance container removed, in accordance with the principles of the present invention;

FIG. 5 illustrates a side perspective of an exemplary embodiment of a fragrance container in accordance with the principles of the present invention;

FIG. 6 illustrates a front perspective of an exemplary embodiment of a fragrance container showing the line, A-A, in which a section view is taken and shown in FIG. 8 in accordance with the principles of the present invention; and

FIG. 7 illustrates a section view of an exemplary embodiment of a fragrance container in accordance with the principles of the present invention.

### DETAILED DESCRIPTION

The fragrance dispensing apparatus, in its many embodiments, provides an apparatus that is used to evenly and effectively disperse a fragrance throughout a room or household to help. Furthermore, the present invention uses a fragrance dispenser, which is attached to a fan, to release controlled amounts of fragrance into the air. The fan is used to improve and control the rate of the dispersion of the fragrance through a house.

The following detailed description is merely exemplary in nature and is not intended to limit the application and uses of the disclosure. As used herein, the word “exemplary” means “serving as an example, instance, or illustration.” Thus, any embodiment described herein as “exemplary” is not necessarily to be construed as preferred or advantageous over other embodiments. Furthermore, there is no intention to be bound by any expressed or implied theory presented in the preceding technical field, background, brief summary, or the following detailed description.

In this description, reference is made to the drawings, wherein like parts are designated with like reference numerals throughout. As used in the description herein and throughout, the meaning of “a,” “an,” and “said” includes plural reference unless the context clearly dictates otherwise. Also, as used in the description herein, the meaning of “in” includes “into” and “on” unless the context clearly dictates otherwise.

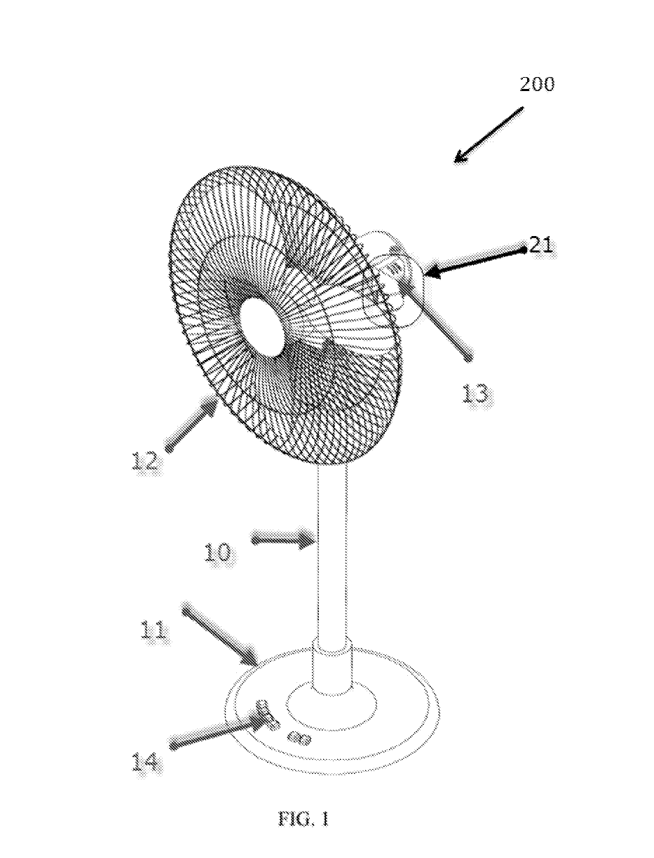

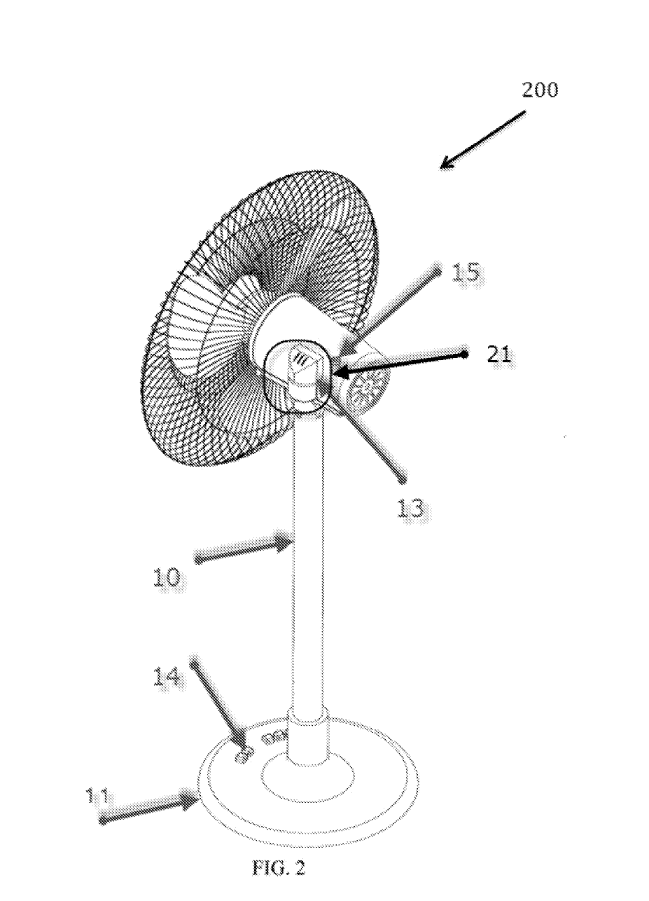

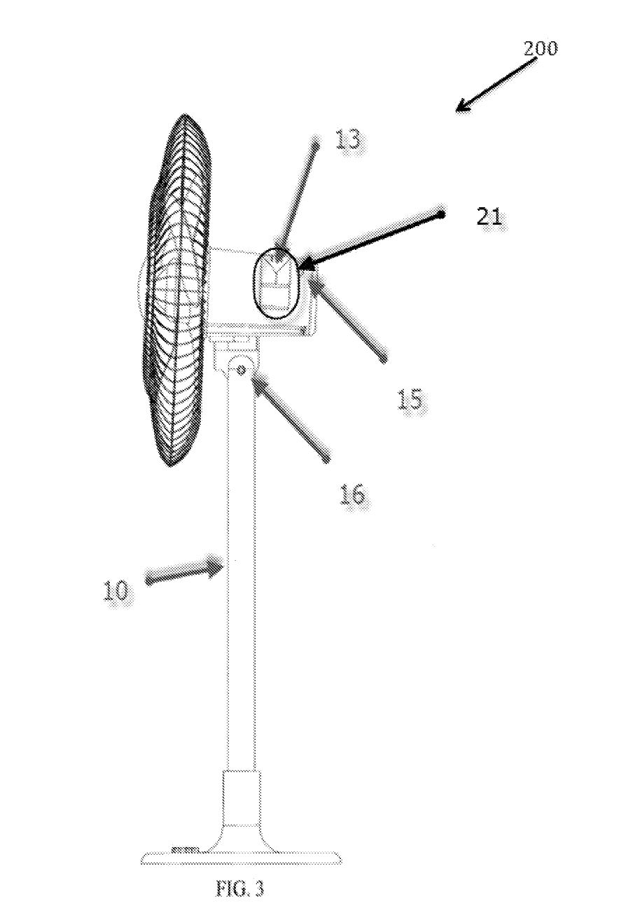

Exemplary embodiments of a fragrance dispensing apparatus 200 are schematically depicted in FIGS. 1-4. Dispensing apparatus 200 includes a common household fan. In an exemplary embodiment, the fan is an oscillating fan to evenly spread a fragrance throughout one or more rooms. In an exemplary embodiment, the fan has three blades which are surrounded by cage 12. In an exemplary embodiment, cage 12 is used to prevent anything from contacting the blades while they are in motion. In alternative embodiments, a plurality of blades is used. In another embodiment, an oscillator mechanism 15 extends from the top of the fan near the back of the fan (as shown in FIGS. 2 and 3). Oscillator mechanism 15 can be pushed or pulled by a user to toggle the fan's oscillating function. In an exemplary embodiment, the fan can be powered by AC provided by standard in-home power outlet.

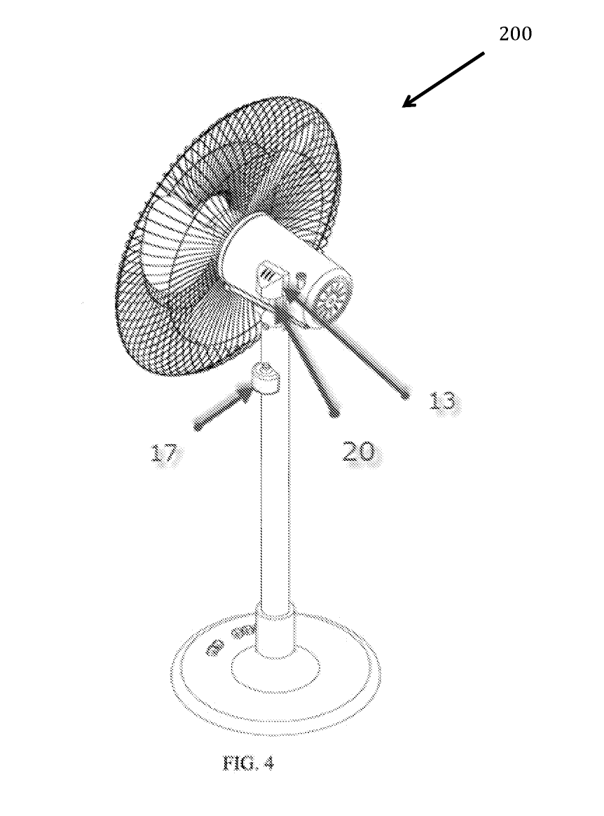

In reference to FIGS. 1-4, fragrance dispenser 21 is used to release an aromatic fragrance into the house. In an embodiment, fragrance dispenser 21 comprises dispensing unit 13 and fragrance container 17 (as shown in FIG. 4). In an exemplary embodiment, dispensing unit 13 is attached to the left side of the fan and is used to direct controlled amounts of fragrance from fragrance container 17 into the air. In an embodiment, dispensing unit 13 comprises a body, a fragrance channel, a heating element, and one or more vents. In another embodiment, the body of dispensing unit 13 is attached to left side of the fan and is used to house the heating element and other electrical components (not shown). In yet another embodiment, the body of dispensing unit 13 is attached to the right side of the fan.

In an exemplary embodiment as shown in FIG. 4, fragrance channel 20 runs through the center of the fragrance dispenser 21 body, creating an opening on the overhanging portion of fragrance dispenser 21 body. In another embodiment, fragrance channel 20 is used to connect fragrance container 17 to dispensing unit 13. In an embodiment, as the fragrance oil inside fragrance container 17 is heated, fragrance channel 20 allows the fragrance to travel to the vents and out into the room. In another embodiment, the heating element is located inside of the body of dispensing unit 13 (not shown) and is used to warm the fragrance oil to the point of evaporation. This causes the fragrance oil to turn into a gaseous state and allow it to be easily spread throughout a room. In one embodiment, one or more vents are located on the top of the body of dispensing unit 13 (not shown). In another embodiment, the vents are small holes in the body of the dispensing unit 13 that allow the fragrance to disperse into the room after it has been warmed in fragrance channel 20 by the heating element (not shown).

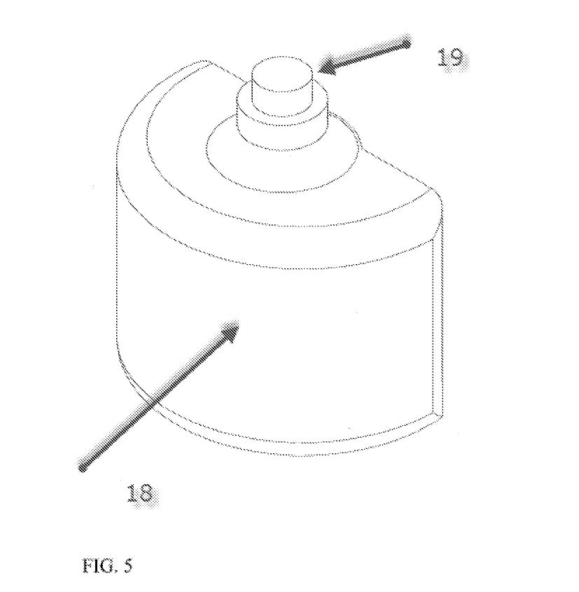

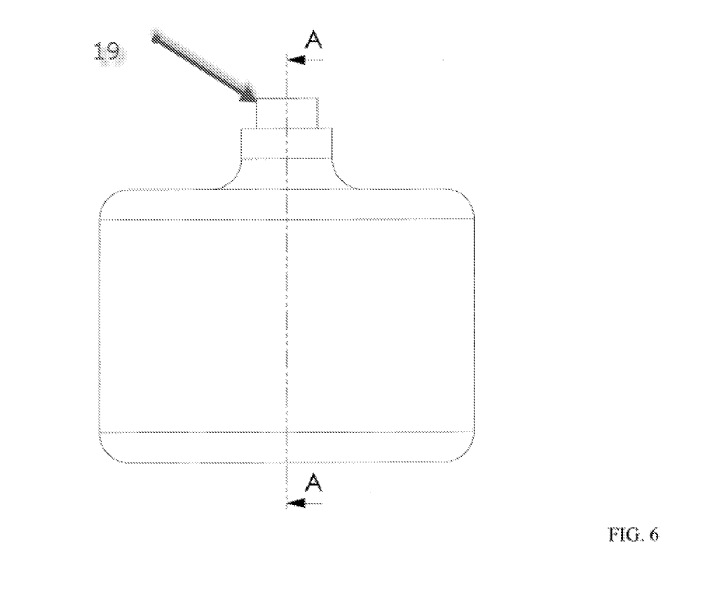

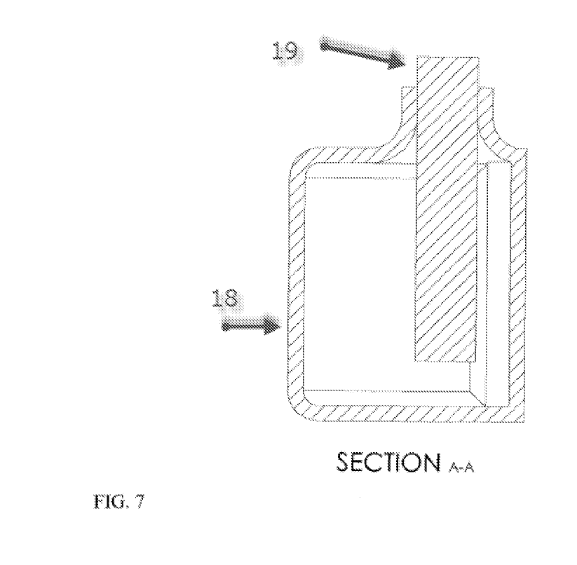

In reference to FIGS. 4-7, fragrance container 17 is used to store a volume of the fragrance oil which is heated and released into the room by the dispensing unit. In an embodiment, fragrance container 17 comprises container body 18, a fragrance chamber, a container opening, and a wick 19 (as shown in FIG. 5). In an embodiment, container body 18 comprises the majority of fragrance container 17 and can be removably connected to dispensing unit 13 via fragrance channel 20. In another embodiment, container body 18 is hollow and comprises glass so a user can easily see the amount of fragrance oil remaining in the container. One of ordinary skill in the art can envision other materials for container body 18 which may vary with the type of fan but not detract from the spirit of the invention.

In reference to FIGS. 4-7, the fragrance chamber is defined by the hollowed portion of container body 18. The hollowed out portion is the area where the fragrance oil is stored. In an embodiment, the container opening is located at the top of container body 18 and provides access to the fragrance chamber from outside of the container body. In another embodiment, wick 19 is a colander of densely packed fibers. In another embodiment, wick 19 extends from the fragrance chamber and protrudes from the top of the container opening. In an embodiment, wick 19 directs the fragrance oil out of the fragrance chamber and into dispensing unit 13 (i.e., through capillary action). In one embodiment, fragrance container 17 may contain different types of fragrances, providing a user different options to choose from. Once a fragrance container runs out of oil, a user may simply connect a full container.

In exemplary embodiments as shown in FIGS. 1-4, fragrance dispensing apparatus 200 comprises a stand to support the fan, and control the functions of the fan and fragrance dispenser 21. In an embodiment, the stand comprises vertical support 10, base 11 and a plurality of buttons 14. In another embodiment, vertical support 10 connects to the bottom of the fan and is used to elevate the fan off the ground. In one embodiment, the connection between the fan and vertical support 10 is a hinged connection 16, which may be locked into place at different angles. In an exemplary embodiment, vertical support 10 is long enough such that it can be placed in the ground but still be able to cool a user. In another exemplary embodiment, vertical support 10 can be shortened, allowing fragrance dispensing apparatus 200 to be placed on a table or desk.

In other exemplary embodiments as depicted in FIGS. 1-4, base 11 of the stand is attached to the bottom of vertical support 10 to keep the fan balanced and prevent it from falling over. This feature is especially necessary if the fan is an oscillating fan. In another embodiment, base 11 is circular in shape; however in alternative embodiments, base 11 is rectangular, or triangular. One of ordinary skill in the art can envision other shapes for base 11 which may vary with the type of fan but not detract from the spirit of the invention. Furthermore, base 11 comprises a number of legs which extends from vertical support 10.

In exemplary embodiments as illustrated in FIGS. 1-4, plurality of buttons 14 is located on top of the surface of base 11. In an embodiment, plurality of buttons 14 are used to control the fan and fragrance dispenser 21. In an exemplary embodiment, plurality of buttons 14 comprise six buttons. In one embodiment, a cluster of four buttons is used to toggle the fan between off, low, medium and high settings. In another embodiment, a second cluster, containing two buttons is used to toggle the fragrance dispenser between on and off settings. Alternatively, the fan and fragrance dispenser 21 comprise more control options. This could include increased options for fan speed, options for controlling the rate in which the fragrance is dispensed and other settings.

Moreover, in alternative embodiments, the fan and fragrance dispenser 21 may be controlled using means other than buttons. This could include but is not limited to knobs, switches, and dials. In one or more embodiments, fragrance dispenser 21 and the fan can be controlled independently so that a user has the option for using the features he/she needs.

In one embodiment, plurality of buttons 14 are located on base 11 of the stand. However, in one or more embodiments, buttons 14 are located on the vertical support 10 or on the oscillating fan, in proximity to the fragrance dispenser 21 of fragrance dispensing apparatus 200. In an alternative embodiment, plurality of buttons 14 are used to control the fan and are located on the top of the fan near its oscillating mechanism 16. In an alternative embodiment, plurality of buttons 14 that are used to control fragrance dispenser 21 are located on dispensing unit 13.

Thus, specific embodiments of a fragrance dispensing apparatus and methods to employ such apparatus have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. An apparatus for dispersing an aromatic fragrance, comprising:
a) a fan;
b) a stand;
c) a base;
d) a fragrance dispenser; and
e) a fragrance container,
wherein:
said oscillating fan is removably connected to said stand;
said fragrance dispenser is removably connected to said oscillating fan; and
said fragrance container is removably connected to said fragrance dispenser; and
wherein said fragrance container and said fragrance dispenser are in fluid communication.

2. The apparatus of claim 1, wherein said fragrance dispenser further comprises a fragrance dispensing unit, wherein said fragrance dispensing unit further comprises a body, a fragrance channel, a heating element, and one or more vents.

3. The apparatus of claim 1, wherein said fragrance container further comprises a body, a fragrance chamber, a container opening, and a wick.

4. The apparatus of claim 1, wherein said oscillating fan further comprises:
a. one or more rotating blades surrounded by a cage, wherein said cage prevents any object from contacting said rotating blades or interfering with the motion of said rotating blades; and
b. an oscillator mechanism extending from the top of said fan, wherein said oscillator mechanism allows a user to control the oscillation of said oscillating fan by manipulating a toggle switch.

5. The apparatus of claim 1, wherein said stand further comprises a mobile, tubular lower frame member having two ends, wherein one of said ends is attached to said oscillating fan with a hinged connection, and wherein said other end of said tubular frame member is attached to a base to vertically support said fan and maintain said fan in a balanced position.

6. The apparatus of claim 2, wherein said stand further comprises a plurality of buttons for controlling:
a) the flow of electrical power to said oscillating fan;
b) the speed of said oscillating fan;
c) the function of said oscillating fan; and
d) the function of said fragrance dispensing unit.

7. The apparatus of claim 2, wherein the body of said fragrance dispenser further comprises a housing for said heating element, wherein said housing is attached to left side of said oscillating fan.

8. The apparatus of claim 2, wherein said fragrance channel further comprises an opening through the center of the body of said fragrance dispenser, creating an opening on the overhanging portion of said body to connect said fragrance container to said fragrance dispensing unit, and said fragrance channel further allows said fragrance to travel to the vents of said fragrance dispenser.

9. The apparatus of claim 2, wherein said vents of said fragrance dispensing unit are located on top of said body of said fragrance dispensing unit, and wherein said vents further comprise small holes to allow dispersal of a fragrance gas created by said heating element of said fragrance dispensing unit.

10. The apparatus of claim 1, wherein the body of said fragrance container is removably connected to said fragrance dispenser via said fragrance channel.

11. The apparatus of claim 1, wherein said fragrance chamber further comprises a hollowed-out portion of the body of said fragrance container for storing a fragrance substance.

12. The apparatus of claim 3, wherein said wick further comprises packed fibers extending from said fragrance chamber, and wherein said wick further protrudes from an opening in said fragrance container to direct said fragrance substance out of said fragrance chamber and into said fragrance dispensing unit through capillary action.
